(19) United States
(12) Patent Application Publication
(10) Pub. No.: US 2006/0212107 A1
(43) Pub. Date: Sep. 21, 2006

(54) Delivery systems, kits and methods for implanting an intraluminal medical device in a body vessel

(76) Inventors: Brian C. Case, Bloomington, IN (US); Jacob Flagle, Indianapolis, IN (US)

Correspondence Address: DUNLAP, CODDING & ROGERS, P.C., P.O. BOX 16370, OKLAHOMA CITY, OK, 73113, US

(21) Appl. No.: 11/376,864

(22) Filed: Mar. 16, 2006

Related U.S. Application Data
(60) Provisional application No. 60/662,838, filed on Mar. 17, 2005.

(51) Int. Cl. A61F 2/06

(52) U.S. Cl. 623/1.11

(57) ABSTRACT

Delivery systems, kits and methods for implanting an intraluminal medical device in a body vessel are provided. Indicia are used on one or more devices to facilitate placement of the intraluminal medical device at a desired point within the body vessel, such as a point at which a size determination has been made.

### CROSS REFERENCE

The present patent application claims priority to the provisional patent application identified by U.S. Ser. No. 60/662,838, filed on Mar. 17, 2005, the entire content of which is hereby expressly incorporated herein by reference.

### FIELD

The present invention relates to medical devices. More particularly, the invention relates to delivery systems, kits and methods for implanting an intraluminal medical device at a point of treatment in a body vessel.

### BACKGROUND

Minimally invasive techniques and instruments for placement of intraluminal medical devices have been developed over recent years and are frequently used to deliver an intraluminal medical device to a desired point of treatment and deploy the intraluminal medical device at the point of treatment. In these techniques, a delivery system is used to carry the intraluminal medical device through a body vessel and to the point of treatment. Once the point of treatment is reached, the intraluminal medical device is deployed from the delivery system for implantation. The delivery system is subsequently withdrawn from the point of treatment and, ultimately, the body vessel. A wide variety of treatment devices that utilize minimally invasive technology have been developed, including stents, stent grafts, occlusion devices, infusion catheters, prosthetic valves, and the like.

In some procedures, the size of the body vessel lumen must be determined prior to implantation of an intraluminal medical device within the body vessel. This enables selection of an appropriately sized intraluminal medical device. Frequently, a sizing catheter is used to determine lumen size in these devices. Following selection of a device based upon the size determination, a delivery system carrying the intraluminal medical device is navigated through the body vessel for implantation.

To minimize the effect of any local variability in vessel lumen size, it is desirable to deploy the intraluminal medical device as near as possible to the point at which the size determination was made. As a result, implantation procedures frequently include steps designed to correlate the sizing and implantation sites. Various techniques can be used, including visualization and anatomical landmarking techniques, to facilitate placement in this manner. Unfortunately, simple changes in the environment, such as patient and/or equipment movement, can complicate the procedure and make precise correlation difficult to achieve.

A need exists for improved devices and techniques that facilitate placement of an intraluminal medical device in a body vessel at a point at which a size determination has been made.

### SUMMARY OF EXEMPLARY EMBODIMENTS

Delivery systems, kits and methods useful for implantation of an intraluminal medical device at a point of treatment in a body vessel are descrobed. Delivery systems, kits and methods according to exemplary embodiments of the invention facilitate placement of an intraluminal medical device at a point in a body vessel at which a size determination has been made.

In one exemplary embodiment, a delivery system comprises an elongate member defining a lumen and a dilator disposed in the lumen. The elongate member defines a window through which a series of indicia, disposed on the dilator, can be viewed. Each indicia of the series of indicia is spaced from a point on a mounting region on the dilator by a predetermined distance. A second series of indicia can be disposed on the elongate member and spaced from the distal end by predetermined distances.

Kits useful in the placement of intraluminal medical devises at a point of treatment in a body vessel are also described. A kit according to one exemplary embodiment comprises a sizing catheter and a delivery system including an elongate member and a dilator that can be disposed in a lumen of the elongate member. A first series of indicia is disposed on the sizing catheter. Each indicia in the first series of indicia is disposed at a predetermined distance from a point on a longitudinal axis of a means for determining vessel size on the sizing catheter. A second series of indicia is disposed on the elongate member of the delivery system. Each indicia of the second series of indicia is disposed at a predetermined distance from the distal end of the elongate member. The predetermined distances of the first series of indicia are substantially the same as the predetermined distances of the second series of indicia.

Methods of implanting a medical device at a point of treatment in a body vessel are also described. One exemplary method according to the invention includes an initial step of providing a sizing catheter and a delivery system. The sizing catheter has a first series of indicia disposed at predetermined distances from a point on a longitudinal axis of a means for determining vessel size and the delivery system includes an elongate member that defines a lumen and includes a second series of indicia disposed at predetermined distances from the distal end of the elongate member. In another step, the sizing catheter is advanced in a body vessel to a point of treatment. In another step, a vessel size determination is made. In another step, the indicia from the first series of indicia corresponding to a particular position is recorded. In another step, the sizing catheter is removed from the body vessel. In another step, the delivery system is advanced in a body vessel until the corresponding indicia from the second series of indicia reaches the position of the recorded indicia. In another step, the delivery system is activated to deploy an intraluminal medical device contained therein. In another step, the delivery system is withdrawn from the body vessel.

The embodiments described briefly above are exemplary in nature. Other delivery systems, kits and methods, including variations on all embodiments described herein, are considered to be within the scope of the claims. Additional understanding of the invention can be obtained by referencing the detailed description of exemplary embodiments of the invention, below, and the appended drawings.

### BRIEF DESCRIPTION OF THE DRAWINGS

FIG. 1 is a perspective view of a delivery system according to one embodiment.

FIG. 2 is an exploded view of the delivery system illustrated in FIG. 1.

FIG. 3 is a perspective view of a sizing catheter that can be used with the delivery system illustrated in FIG. 1.

FIG. 4 is a perspective view of a delivery system according to another exemplary embodiment.

FIG. 5 is an exploded view of the delivery system illustrated in FIG. 4.

FIG. 6A is a perspective view of the delivery system illustrated in FIG. 4 in a first stage of deployment.

FIG. 6B is a perspective view of the delivery system illustrated in FIG. 4 in a second stage of deployment.

FIG. 6C is a perspective view of the delivery system illustrated in FIG. 4 in a third stage of deployment.

FIG. 7 is a schematic illustration of a kit according to an embodiment.

FIG. 8 is a flow diagram illustrating a method according to the invention.

### DETAILED DESCRIPTION OF EXEMPLARY EMBODIMENTS

The following detailed description and appended drawings describe and illustrate various exemplary embodiments. The description and drawings serve to enable one skilled in the art to make and use the invention, and are not intended to limit the scope of the invention, or its protection, in any manner.

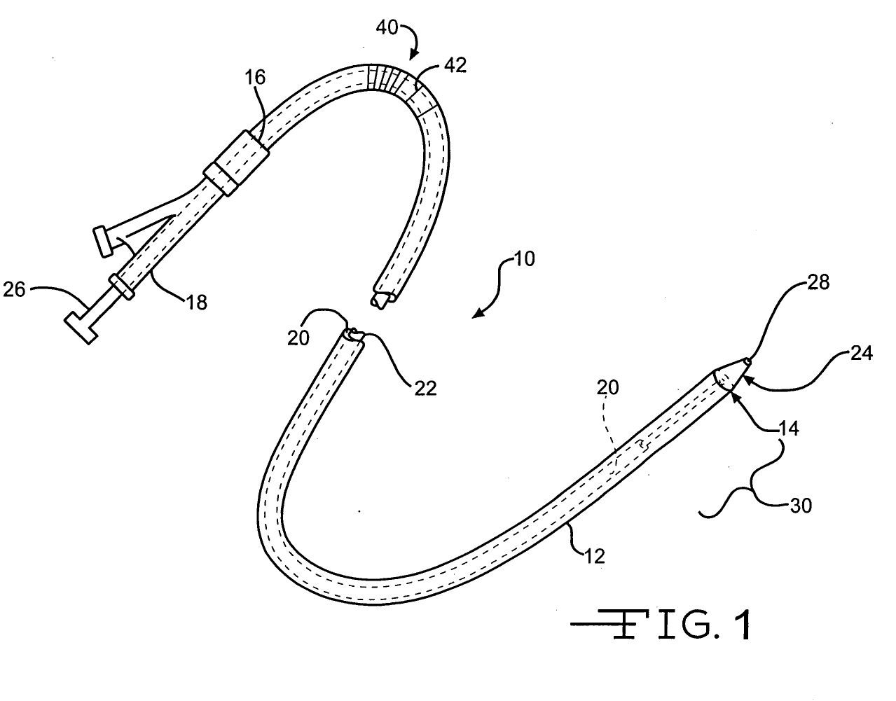

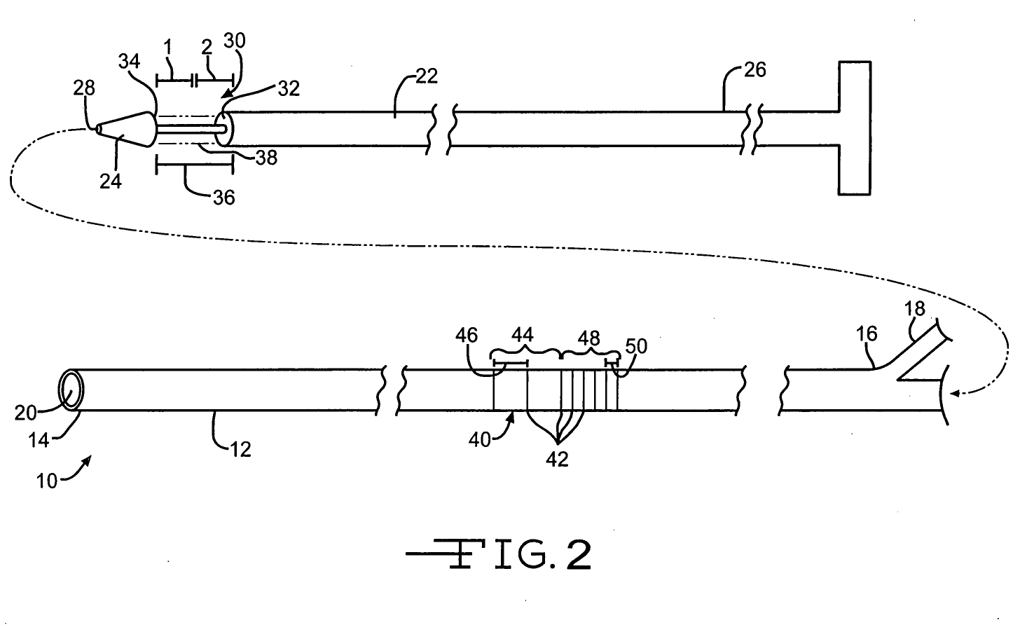

FIGS. 1 and 2 illustrate a delivery system 10 according to a first exemplary embodiment. The delivery system 10 includes an elongate tubular member 12 having a distal end 14 which is insertable in a body vessel and a proximal end 16 that can be coupled to a connector 18, such as a Touhy-Borst adaptor or other suitable functional component. The tubular member 12 defines a lumen 20 extending between the distal 14 and proximal 16 ends and acts as a sheath for components disposed therein.

The delivery system 10 includes a dilator 22 disposed within the lumen 20 of the tubular member 12. As used herein, the term “dilator” refers to an elongate member capable of being disposed within a lumen of a sheath, such as tubular member 12. The dilator 22 has a distal end 24 that is insertable in a body vessel and a proximal end 26. The distal end 24 advantageously defines a tapered edge that terminates in an opening. The dilator 22 defines a wire guide lumen 28 that extends between the opening at the distal end 24 and the proximal end 26. The wire guide lumen 28 accommodates a wire guide (not illustrated), allowing the delivery system 10 to be used in standard percutaneous techniques. While the illustrated embodiment is adapted for over-the-wire techniques, i.e., the wire guide lumen 28 extends between the proximal 26 and distal 24 ends of the dilator 22, it is understood that a rapid exchange type wire guide lumen could also be used. Thus, the wire guide lumen 28 can extend along only a portion of the length of the dilator 22. Indeed the wire guide lumen 28 can extend along any suitable length of the dilator 22. In these alternate embodiments, the dilator 22 advantageously includes a first opening on the distal end 24 of the dilator and a second opening in a sidewall of the dilator 22. Both openings provide access to the wire guide lumen 28.

The dilator 22 defies a mounting region 30 having proximal 32 and distal 34 ends and a length 36 extending from the proximal end 32 to the distal end 34. An expandable intraluminal medical device 38 is disposed around a portion of the dilator 22 at the mounting region 30 between the proximal 32 and distal 34 ends. The intraluminal medical device 38 may be any suitable intraluminal medical device known in the art or hereinafter developed. Examples of suitable types of intraluminal medical devices include stents, prosthetic valves, filters, and the like. Further, the intraluminal medical device 38 can be a self-expandable device or a device that requires an input of force for expansion, such as a balloon-expandable device.

Suitable self-expandable intraluminal medical devices include those described in U.S. Pat. No. 6,200,336 to Pavcnik et al. for a MULTIPLE-SIDED INTRALUMINAL MEDICAL DEVICE; U.S. application for patent Ser. No. 10/642,372 of Pavcnik et al. for an IMPLANTABLE VASCULAR DEVICE, filed on Aug. 15, 2003; and U.S. application for patent Ser. No. 10/828,716 of Case et al. for an ARTIFICIAL VALVE PROSTHESIS WITH IMPROVED FLOW DYNAMICS, filed on Apr. 21, 2004; the entire disclosures of which are hereby incorporated into this disclosure for the purpose of describing suitable intraluminal medical devices for use in embodiments of the present invention.

The tubular member 12 includes a series 40 of indicia 42. Each of the indicia 42 is disposed on the tubular member at a pre-determined distance from the distal end 14 of the tubular member 12. Thus, each of the indicia 42 corresponds to a pre-determined distance from the distal end 14 of the tubular member 12. In the embodiment illustrated in FIGS. 1 and 2, the indicia in a first set 44 of indicia 42 are separated from each other by a first distance 46. The indicia in a second set 48 of indicia 42 are separated from each other by a second distance 50.

Any suitable type and number of indicia can be used. Examples of suitable types of indicia include markings on the external surface and markers disposed in the material of the tubular member 12. As described below, the indicia 42 provide positioning information during an implantation procedure. Accordingly, the indicia chosen need only be able to convey positioning information to a user. While visual indicia are illustrated in FIGS. 1 and 2, it is contemplated that indicia that convey information through other senses, including touch, can be used.

Each of the indicia 42 can be disposed at any suitable distance from the distal end 14 of the tubular member 12. The specific distances chosen will depend on several considerations, including the overall length of the delivery system 10, the type and/or nature of the intraluminal medical device 38, and the nature of the body vessel in which the delivery system 10 will be used. For venous applications, such as delivery systems for implantation of valve devices in human veins, a first series of indicia starting at approximately 70 mm from the distal end 14 and separated by a distance of 10 mm is suitable. A second series of indicia starting at approximately 100 mm from the distal end 14 and separated by a distance of 2 mm can also be used in combination with the first series.

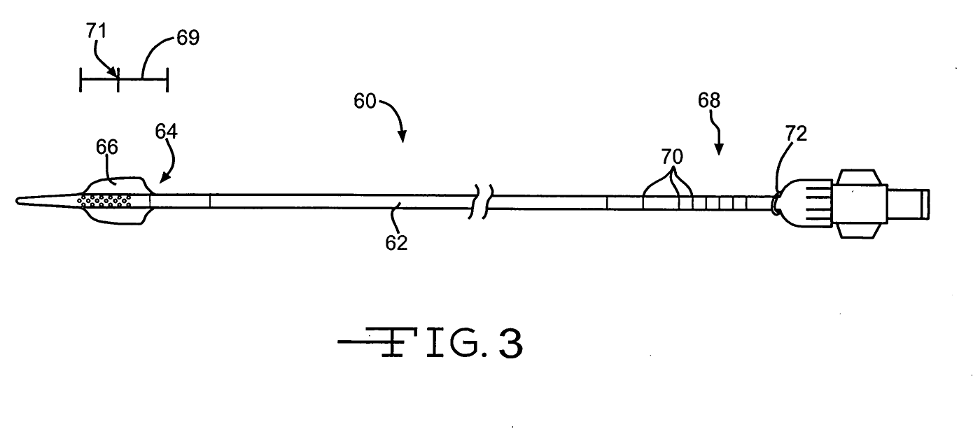

FIG. 3 illustrates a sizing catheter 60 that can be used with the delivery system 10 illustrated in FIGS. 1 and 2. The sizing catheter 60 includes a main body 62 and a means for determining vessel lumen size 64. Any suitable means for determining vessel lumen size can be used, including an inflatable balloon 66, an expandable basket, a laser-based measuring system, and other suitable structure. The sizing catheter 60 includes a series 68 of indicia 70. Each of the indicia 70 is disposed at a pre-determined distance from a predetermined point along the length 69 of the means for determining vessel lumen size 64, such as an approximate midpoint 71 on a lengthwise axis of the means for determining vessel lumen size 64.

The pre-determined distance for each indicia 70 on the sizing catheter 60 corresponds to the pre-determined distance for an indicia 42 on the tubular member 12 of the delivery system 10 (measured from the distal end 14). That is, the pre-determined distance for an indicia 70 of the series 68 of indicia 70 or the sizing catheter 60 is substantially the same as the pre-determined distance for one indicia 42 of the series 40 of indicia 42 or the tubular member 12. The two series 40, 68 of indicia can include one or more characteristics that indicate this correspondence of distances. For example, each indicia 42 on the elongate member 12 can have a unique color or pattern and the corresponding indicia 70 on the sizing catheter 60 can have the same color or pattern. Any suitable means for indicating a correspondence between indicia on separate members can be used, including visual and other suitable means.

The sizing catheter 60 can include an optional marking memberthat can be moved along the main body 62 to mark a particular indicia 70 of interest. Any suitable movable member can be used as the marking member. As illustrated in FIG. 3, a ring member 72, such as an elastomeric or polymeric ring member, that surrounds the main body 62 can be used. A member that can be temporarily secured at or near an indicia 70, such as an elastomeric ring member that is moveable along the main body 62 but that can be temporarily secured on the main body 62, is considered advantageous.

In an implantation procedure, the sizing catheter 60 is navigated through a body vessel until the means for determining vessel lumen size 64 reaches a desired point of treatment within the body vessel. Once that point is reached; the means for determining vessel lumen size 64 is activated and sizing information is determined. Outside of the patient, the identity and position of one or more indicia 70 relative to the point of entry, or another suitable position, is recorded. For example, the identity of the indicia 70 positioned immediately adjacent the point of entry can be recorded. The marking member 72 can be moved to the indicia 70 of interest for later reference, eliminating any uncertainty in later steps. Following the size determination, the sizing catheter 60 is withdrawn from the body vessel. The delivery system 10 is then inserted into the body vessel and navigated to the point of treatment. This navigation step can be conducted by moving the delivery system 10 along a previously placed wire guide by passing the wire guide through the wire guide lumen 28 of the dilator 22. The delivery system 10 is advanced within the body vessel until the indicia 42 spaced from the distal end 14 by the same distance as the identified indicia 70 on the sizing catheter 60 is spaced from the means for determining vessel lumen size 64 reaches the position of the identified indicia 70 at the time of its identification (e.g., immediately adjacent the point of entry). This can be accomplished relatively quickly if a means for indicating a correspondence between indicia is included, such as a color-coding scheme. For example, if each of the indicia 70 on the sizing catheter 60 has a unique color and the series 40 of indicia 42 on the elongate member has a corresponding color scheme, the delivery system can be advanced until the indicia 42 with the appropriate color reaches the position of the identified indicia 70 of the sizing catheter 60. Once the corresponding indicia is at the position (e.g., immediately adjacent the point of entry), the user is assured that the distal end 14 of the elongate member 12 is at the point at which the size determination was made. The elongate member 12 can then be retracted in the normal fashion to deploy the intraluminal medical device 38.

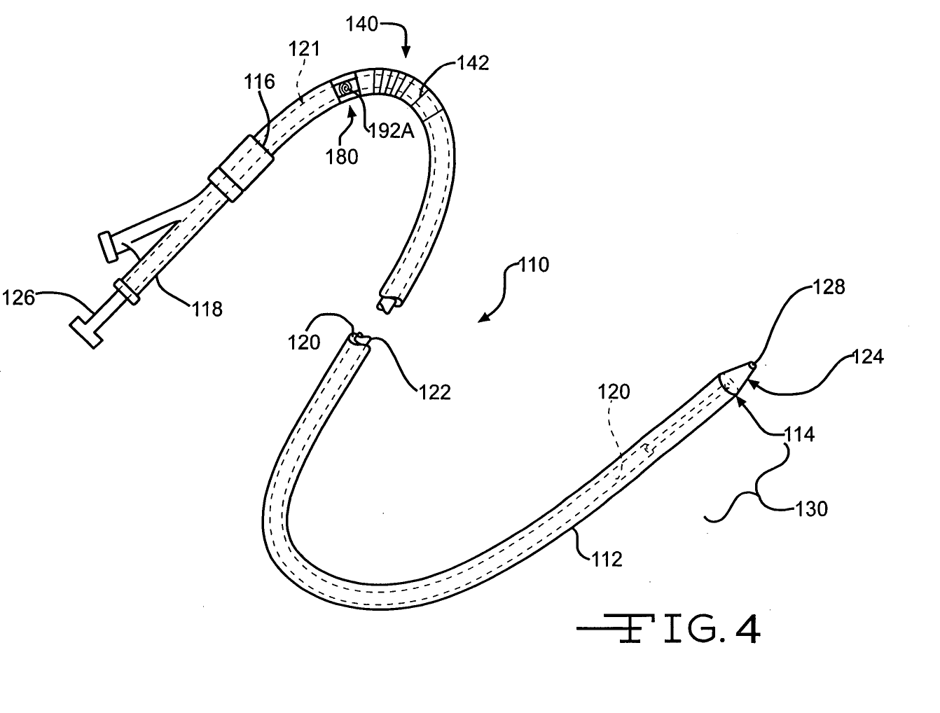

FIGS. 4 and 5 illustrate a delivery system 110 according to a second exemplary embodiment. The delivery system 110 according to this embodiment is identical to the embodiment illustrated in FIGS. 1 and 2, except as described below. Thus, similar features and components have similar reference numbers, increased by 100.

In this embodiment, the elongate member 112 includes a window 180 through which the dilator 120 can be visualized. As best illustrated in FIG. 5, the dilator 120 includes a series 190 of markings 192A, 192B, 192C, 192D, each of which are positioned at a predetermined distance from a point in the mounting region 130, such as the distal end 134, and can be visualized through the window 180 of the elongate member 112 when positioned adjacent the window 180.

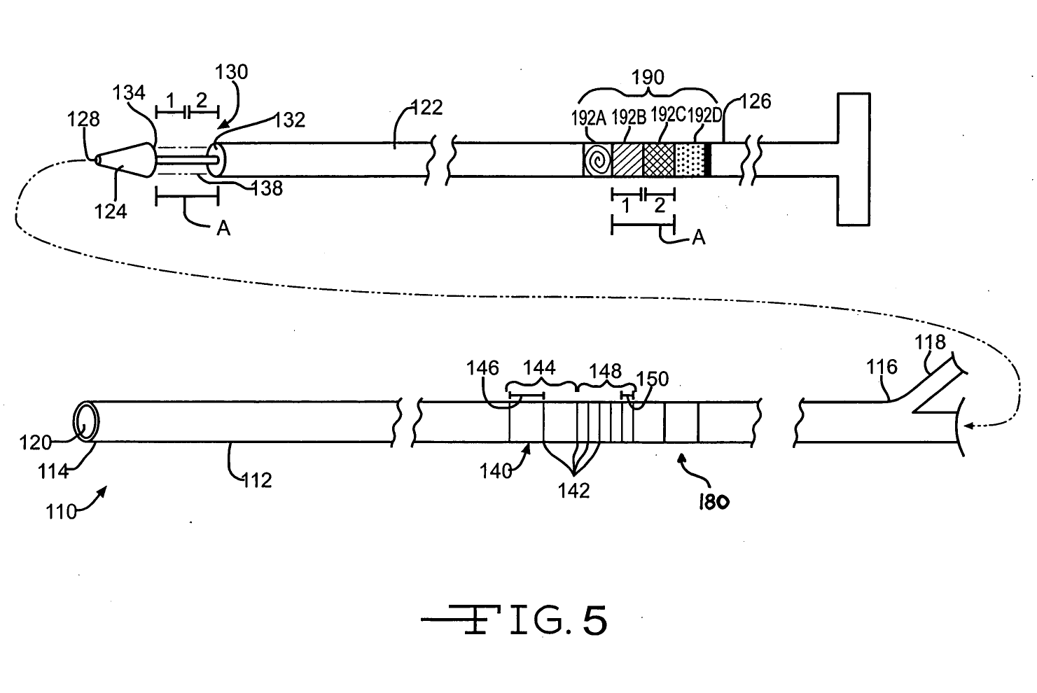

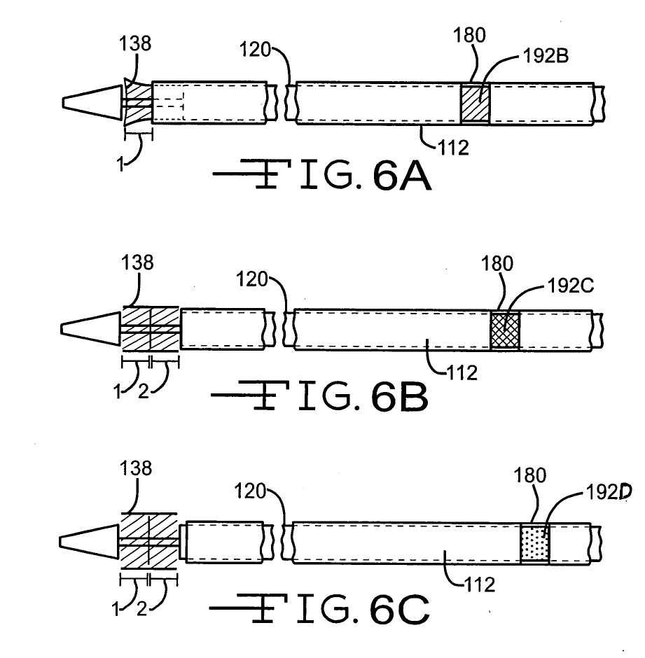

Each of the markings 192A, 192B, 192C, 192D, when viewed through the window 180, conveys information relating to the deployment status of the intraluminal medical device 138. That is, each of the markings 192A, 192B, 192C, 192D, when viewed through the window 180, conveys information relating to the relative positioning between a distal portion of the elongate member 112 and the mounting region 130 of the dilator 120. As best illustrated in FIG. 4, the first marking 192A of the illustrated embodiment, when viewed through the window 180, indicates that the elongate member 112 is disposed over the mounting region 130 and the intraluminal medical device 138. In this position, the user knows that deployment of the intraluminal medical device 138 has not been initiated. FIG. 6A illustrates a first stage of deployment in which the second marker 192B is viewable through the window 180. In this configuration, the user knows that deployment has begun for at least a first portion 1 of the intraluminal medical device 138. FIG. 6B illustrates a second stage of deployment in which the third marker 192C is viewable through the window 180. In this configuration, the user knows that deployment of a second portion 2 of the intraluminal medical device 138 has begun. FIG. 6C illustrates a third stage of deployment in which the fourth marker 192D is viewable through the window 180. In this configuration, the user knows that the tubular member 112 is completely withdrawn from its position over the mounting region 130 and that the intraluminal medical device 138 is completely deployed in the case of a self-expandable medical device 138. At this point, withdrawal of the delivery system 110 can be initiated.

The window 180 can be formed in any particular manner, such as by removing a portion of the material of the elongate member 112 at a desirable location and by including transparent of other suitable material at a desired location.

This embodiment is particularly advantageous for use with intraluminal medical devices having multiple stages. For example, an intraluminal medical device with a first stage that lacks barbs or other means for anchoring the device and a second stage that includes barbs or other such means for anchoring. By using the delivery system 110 to deploy the first stage, a user can verify positioning of the intraluminal medical device prior to deploying the second, anchoring stage. Verification of position prior to final anchoring of an intraluminal medical device is advantageous for the placement of intraluminal medical devices the functioning of which may be sensitive to relative position within a body vessel, such as prosthetic valves, including prosthetic heart valves and prosthetic venous valves.

Using the delivery system according to this embodiment, a user can retract the tubular member 112 to a first position, indicated by viewing the second marking 192B through the window 180, to deploy the first stage. If the first stage is the stage that lacks barbs or other means for anchoring, the user is assured that repositioning of the device can be conducted with minimal engagement of the vessel wall. Once a desired positioning is achieved, the elongate member can be retracted to a second position, indicated by viewing the third marking 192C through the window 180. At this point, the user is aware that the second stage of the device has been deployed. If the second stage includes barbs or other means for anchoring, the device will be anchored on the vessel. Finally, the tubular member can be retracted to a third position, indicated by viewing the fourth marking 192D through the window 180. At this point, the user is aware that the intraluminal medical device has been fully deployed and retraction of the delivery system 110 from the body vessel can be initiated.

The first series 140 of indicia 142 are considered optional in this embodiment. Inclusion of these indicia 142 is advantageous when the delivery system 110 is to be used with a sizing catheter or other device that includes a corresponding set of indicia.

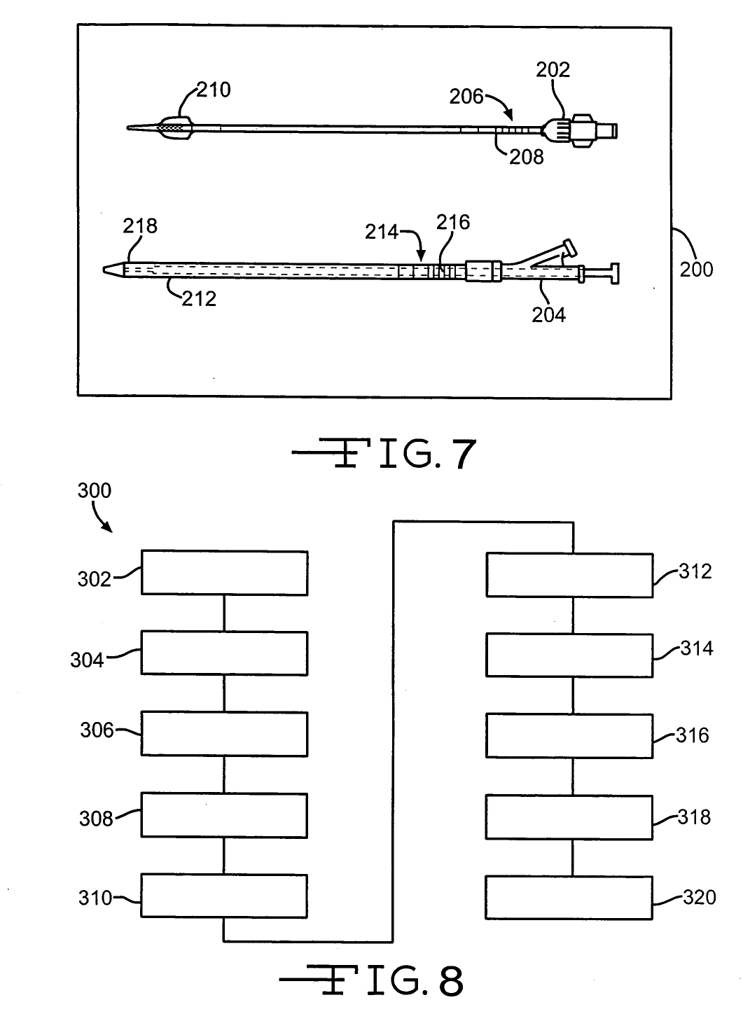

FIG. 7 illustrates a kit 200 according to an exemplary embodiment of the invention. The kit 200 includes a sizing catheter 202 and a delivery system 204. The sizing catheter 202 includes a series 206 of indicia 208, each of which is spaced a predetermined distance from a point, such as a midpoint, on a longitudinal axis of a means for determining vessel lumen size 210, as described above. The elongate member 212 of the delivery system 204 includes a series 214 of indicia 216, each of which is spaced a predetermined distance from the distal end 218 of the elongate member 212 that is substantially the same as the predetermined distance for a corresponding indicia 208 in the series 206 of indicia 208 on the sizing catheter 202. The sizing catheter 202 and the delivery system 204 can each be in accordance with any embodiment of the invention. The series 206 of indicia 208 on the sizing catheter 202 and the series 214 of indicia 216 on the elongate member 212 each advantageously include a means for indicating a correspondence between indicia on separate members, such as a common color scheme or common patterns.

The kits according to the invention provide a useful implement for accurately placing an intraluminal medical device substantially at a point in a body vessel at which a size determination has been made.

FIG. 8 illustrates an exemplary method 300 for implanting an intraluminal medical device according to the invention. In an initial step 302, a sizing device is provided. The sizing device includes a first series of indicia disposed at predetermined distances from a point on a longitudinal axis of a means for determining vessel lumen size. In another step 304, a delivery system is provided. The delivery system includes an elongate member, a dilator disposed within a lumen of the elongate member, and an intraluminal medical device disposed on a mounting region of the dilator. The elongate member includes a second series of indicia disposed at predetermined distances from the distal end of the elongate member. The predetermined distances of the first set of indicia are substantially the same as the predetermined distances of the second set of indicia.

In another step 306, the sizing device is inserted into a body vessel to a point of treatment. In anther step 308, a size determination is made, such as a vessel lumen size determination, using the sizing device. In another step 310, the indicia in the first series of indicia that corresponds to a particular position, such as adjacent the point of entry, is recorded. In another step 312, the sizing device is removed from the body vessel. In another step 314, the delivery system is inserted into the body vessel. In another step 316, the delivery system is advanced in the body vessel until the indicia from the second set of indicia that has the same predetermined distance as the indicia recorded in step 310 is positioned at the position used in step 310, such as adjacent the point of entry. In another step 318, the intraluminal medical device is deployed. In another step 320, the delivery system is withdrawn from the body vessel.

The foregoing detailed description provides exemplary embodiments of the invention and includes the best mode for practicing the invention. These embodiments are intended only to serve as examples of the invention, and are not intended to limit the scope of the invention, or its protection, in any manner.

What is claimed is:

1. A kit for implanting an intraluminal medical device in a body vessel, comprising:
a sizing catheter comprising a means for determining lumen size of said body vessel and a first series of indicia, each indicia of the first series of indicia spaced from a point on a longitudinal axis of the means for determining lumen size of said body vessel by a predetermined distance; and
a delivery system comprising an elongate member defining a lumen and a dilator disposed in the lumen, the elongate member having a distal end and a second series of indicia, each indicia of the second series of indicia spaced from the distal end by a predetermined distance that is substantially the same as a predetermined distance for an indicia of the first series of indicia.

2. The kit for implanting an intraluminal medical device according to claim 1, wherein the means for determining lumen size of said body vessel comprises a balloon.

3. The kit for implanting an intraluminal medical device according to claim 1, wherein the point on a longitudinal axis of the means for determining lumen size of said body vessel comprises a midpoint.

4. The kit for implanting an intraluminal medical device according to claim 1, wherein the first series of indicia comprises first and second sets of indicia, each indicia of the first set of indicia spaced from another indicia of the first set of indicia by a first distance and each indicia of the second set of indicia spaced from another indicia of the second set of indicia by a second distance.

5. The kit for implanting an intraluminal medical device according to claim 1, wherein the second series of indicia comprises first and second sets of indicia, each indicia of the first set of indicia spaced from another indicia of the first set of indicia by a first distance and each indicia of the second set of indicia spaced from another indicia of the second set of indicia by a second distance.

6. The kit for implanting an intraluminal medical device according to claim 1, wherein the dilator includes a distal tip and a third series of indicia, each indicia of the third series of indicia spaced from the distal tip by a predetermined distance.

7. The kit for implanting an intraluminal medical device according to claim 6, wherein the elongate member comprises a window through which at least one indicia of the third series of indicia can be viewed.

8. The kit for implanting an intraluminal medical device according to claim 1, wherein the sizing catheter comprises a marking member adapted to be moved along the sizing catheter to mark an indicia of the first series of indicia.

9. The kit for implanting an intraluminal medical device according to claim 8, wherein the marking member is disposed about a portion of the sizing catheter.

10. The kit for implanting an intraluminal medical device according to claim 9, wherein the marking member comprises an elastomeric ring member.

11. The kit for implanting an intraluminal medical device according to claim 1, wherein the dilator defines a mounting region and an expandable intraluminal medical device is disposed in the mounting region.

12. The kit for implanting an intraluminal medical device according to claim 11, wherein the expandable intraluminal medical device includes first and second stages, the second stage comprising a means for anchoring the expandable intraluminal medical device within a body vessel.

13. The kit for implanting an intraluminal medical device according to claim 11, wherein the expandable intraluminal medical device comprises a prosthetic valve.

14. A delivery system for implanting an expandable intraluminal medical device in a body vessel, comprising:
a dilator defining a mounting region for receiving said implantable medical device, and a series of indicia, each indicia of the series of indicia spaced from a point on a lengthwise axis of the mounting region by a predetermined distance; and
an elongate member defining a lumen for slidably receiving the dilator, and having a window, at least one indicia of the series of indicia being viewable through the window when disposed adjacent the window.

15. The delivery system for implanting an expandable intraluminal medical device according to claim 14, wherein said expandable intraluminal medical device includes first and second axial stages; and
wherein a first indicia of the series of indicia has a length substantially equal to a length of the first stage.

16. The delivery system for implanting an expandable intraluminal medical device according to claim 15, wherein a second indicia of the series of indicia has a length substantially equal to a length of the second stage.

17. A method of implanting an expandable intraluminal medical device, comprising:
providing a sizing device comprising a means for determining vessel lumen size in a first series of indicia, each indicia of the first series of indicia spaced from a point on a longitudinal axis of the means for determining vessel lumen size by a predetermined distance; and
providing a delivery system comprising an elongate member defining a lumen, a dilator disposed in the lumen, and a second series of indicia, each indicia of the second series of indicia spaced from a distal end of the elongate member by a predetermined distance that is substantially the same as a predetermined distance of an indicia of the first set of indicia; inserting the sizing device into a body vessel;
making a vessel size determination;
recording an indicia of the first series of indicia that is disposed adjacent a point of interest;
removing the sizing device from the body vessel;
inserting the delivery system into the body vessel;
advancing the delivery system in the body vessel until an indicia of the second series of indicia having substantially the same predetermined distance as the indicia of the first series of indicia identified in the recording step is disposed adjacent the point of interest;
deploying the expandable intraluminal medical device;
withdrawing the delivery system from the body vessel.

18. The method of implanting an expandable intraluminal medical device according to claim 17, wherein the expandable intraluminal medical device comprises a prosthetic valve.
